(19) United States
(12) Patent Application Publication
(10) Pub. No.: US 2012/0065137 A1
(43) Pub. Date: Mar. 15, 2012

(54) THERAPEUTICS FOR TRAUMA INDUCED FACTOR V CONSUMPTIVE COAGULOPATHY

(76) Inventors: Richard Jenny, Essex Junction, VT (US); Kenneth G. Mann, Grand Isle, VT (US)

(21) Appl. No.: 13/225,089

(22) Filed: Sep. 2, 2011

Related U.S. Application Data
(60) Provisional application No. 61/380,122, filed on Sep. 3, 2010.

(51) Int. Cl. A61K 38/36; A61P 7/04

(52) U.S. Cl. 514/14.7; 514/13.7

(57) ABSTRACT

A process of treating trauma induced factor V consumptive coagulopathy is presented whereby a subject is administered a preparation of isolated factor V or a variant thereof. Administration of factor V surprisingly improves clot times and reduces the severity and propensity of bleeding events.

CROSS REFERENCE TO RELATED APPLICATIONS

This application depends from and claims priority to U.S. Provisional Application No. 61/380,122, filed Sep. 3, 2010, the contents of which are incorporated herein by reference in their entirety.

### FIELD OF THE INVENTION

The invention relates to therapeutics for trauma related coagulation factor V consumption. Specifically, the invention provides therapeutic factor V or factor V related preparations that address symptoms of factor V consumption in patients suffering trauma such as impact, surgical, inflammatory, or shock traumas.

### BACKGROUND OF THE INVENTION

Hemostasis is a delicate balance of procoagulant and anti-coagulant activity. Small deviations from this balance can lead to severe bleeding to thrombotic complications. Central to proper hemostasis is the production of thrombin. Thrombin serves as the primary procoagulant protease cleaving fibrinogen to fibrin leading to clot formation. As a procoagulant molecule, thrombin also creates a positive feedback loop furthering its own production. The small amount of thrombin that is generated during the initiation phase of blood coagulation, proteolytically converts factor V to the active cofactor, factor Va. The newly formed factor Va combines with factor Xa in a calcium-dependent manner on a phospholipid surface to form the prothrombinase complex. The prothrombinase complex is responsible for the rapid and extensive thrombin production that occurs during the propagation phase of blood coagulation.

Interestingly, thrombin is also responsible for downregulating its own production. Thrombin activates protein C to activated protein C (APC). APC is the primary protease responsible for inactivation of the prothrombinase complex by cleaving factor Va, thus, inactivating the complex. As such, the level of factor V in the system, and factor Va available for incorporation into prothrombinase, serves as a central regulator of thrombin production and clot formation.

Trauma induced coagulopathy is a rare complication of severe trauma. Patients presenting with trauma induced coagulopathy suffer severe bleeding which can result in death without the appropriate medical intervention. In approximately 20% of patients presenting trauma induced coagulopathy the level of circulating intact factor V is depleted. This depleted level of circulating factor V reduces the amount of cofactor available for participation in prothrombinase thereby reducing thrombin production and clot formation.

Trauma induced Factor V consumptive coagulopathy, while resulting in bleeding, is distinguishable from other conditions such as hemophilia, factor V deficiency, and acquired factor V inhibitors in the factor V depletion may be the result of increased proteolytic or other degradative activity specific for one or more soluble coagulation factors. Traditional therapies such as fresh frozen plasma have numerous undesirable side effects or complications. Thus, there is a need for processes and therapeutics to treat subjects presenting with trauma induced factor V consumptive coagulopathy.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

A process for treating trauma induced factor V consumptive coagulopathy is presented whereby a subject is administered an isolated factor V protein or variant thereof, or an oligonucleotide encoding factor V or a variant thereof. The administration reduces the severity or propensity of bleeding events, decreases the clot time and/or thrombin time, increases the clot strength, or prolongs clot dissolution time, or increases the circulating levels of factor V in a subject's plasma.

The factor V is illustratively substantially inactive prior to said administering. The factor V or variant thereof may have a non-wild-type amino acid substitution, deletion, or addition that renders factor V or factor Va resistant to cleavage by a protease. Optionally, a non-wild-type amino acid is present at a protease cleavage site within the factor V. Typical locations for non-wild-type or modified amino acids are at or near a protease cleavage site. Other locations are in regions of factor V that interact with or modulate interactions with proteases, or cofactors of proteolysis. Typical protease cleavage resistance is resistance to cleavage by plasmin, cathepsin G, elastase, a platelet derived protease, activated protein C, or combinations thereof.

A factor V molecule or variant thereof is optionally cleaved at Arg 709, Arg 1018, Arg 1545, or combinations thereof prior to said administration.

A factor V molecule or variant thereof is optionally recombinantly expressed.

An inventive process illustratively includes obtaining a biological sample from a subject following trauma and quantifying post-trauma factor V antigen in the biological sample. This level of factor V is optionally compared to the level of factor V present in a biological sample from a subject obtained prior to trauma whereby pre-trauma factor V antigen in said first biological sample is quantified. Alternatively, the subjects level of factor V is compared to the level of factor V expected to be present in physiologically normal plasma. This comparison allows diagnosis of factor V consumptive trauma induced coagulopathy in the subject.

Optionally, a subject has at least one copy of a gene encoding wild-type factor V.

### DETAILED DESCRIPTION OF THE PREFERRED EMBODIMENTS

The following description of the preferred embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention, but are presented for illustrative and descriptive purposes only.

Processes and compositions are presented to ameliorate the severity of, treat, cure, or otherwise alter a symptom of trauma induced factor V consumptive coagulopathy. The invention has utility as a therapy for bleeding in mammalian subjects. The acute coagulopathy presented by trauma induced factor V consumptive coagulopathy may be specific for a subset of coagulation factors. Brohi, K, et al., Curr Opin Crit Care, 2007; 13:680-685, incorporated herein by reference, indicate that activated Protein C (APC) may play a role in the condition. Cleavage activity of APC in general would be expected to deplete factor VIII as well as factor V. The invention provides a method of addressing acute coagulopathy in an identified subset of subjects characterized by a principle factor V deficiency, or factor V deficiency alone, by administration of isolated factor V, isolated factor Va, or an isolated factor V(a) variant that is resistant to protease cleavage, without the need for administration of fresh frozen plasma.

The invention provides a process for treatment of trauma induced factor V consumptive coagulopathy. In some instances of trauma induced coagulopathy (TIC) disseminated intravascular coagulation (DIC) occurs forming numerous clots throughout a subject's vasculature. While DIC can ultimately lead to factor depletion, factor V consumptive TIC presents factor V consumption that is presumably mediated by a protease or other related degradation of soluble factor V in the blood which results in bleeding. In some embodiments the factor V consumptive TIC presents with depletion of one or more other coagulation factors that are insufficient alone to produce bleeding. Optionally, the depletion of other coagulation factors is less than 20% to less than 5% inclusive. In some embodiments, depletion of multiple coagulation factors sufficient to induce bleeding occurs along with depletion of factor V. The inventors expect that administration of factor V according to the present invention in the absence of other plasma factors is may reduce the magnitude of bleeding. Other coagulation factors include soluble and membrane bound proteins involved in procoagulant or anticoagulant cascades. Illustrative examples of other coagulation factors include prothrombin, factor VII, factor VIII, tissue factor, factor IX, factor X, factor XI, factor XII, factor XIII, TAFI, plasminogen, or activated forms thereof. In some embodiments, the level of one or more other coagulation factors is depleted to such an extent that bleeding from the depletion is expected. One of skill in the art recognizes how to determine such levels of other coagulation factors. The administration of isolated soluble factor V surprisingly ameliorates the bleeding symptoms of factor V consumptive TIC independent on whether other coagulation factors are depleted or not and promotes the return to normal coagulation in a subject.

It is appreciated that factor V consumptive TIC is optionally diagnosed by the presence of a known trauma such as a physical impact, blast, percussion, surgical procedure, gunshot wound, inflammation, hypothermia, hyperthermia, or other recognized trauma combined with a quantified level of factor V in a biological sample that is less than 30% the levels present in a normal population.

An inventive process includes administering and effective amount of isolated factor V protein or a variant thereof to a subject presenting with TIC. A “subject” as defined herein is illustratively a mammal. A subject is illustratively a primate including higher and lower primates. Higher primates illustratively include humans. Optionally, a subject is a rodent. A rodent illustratively includes a rat or mouse. Other subjects illustratively include a hamster, guinea pig, pig, horse, sheep, bovine, donkey, dog, or cat. A subject is optionally an organism suspected of or having a trauma. A subject is optionally a patient.

As used herein the term “trauma” is illustratively: impact trauma such as that resulting from a vehicle accident, gunshot wound, percussive impact, surgery, and the like; inflammation; shock; cancer; obstetric trauma illustratively pre-eclampsia, abruption placentae, or amniotic fluid embolism; burns; heat stroke; and infections such as bacterial infections illustratively gram-negative sepsis leading to rocky mountain spotted fever, and viral infections illustratively those causing hemorrhagic fever. It is appreciated that this is a limited list, and other traumas leading to factor V consumptive TIC are similarly recognized by those of skill in the art as applicable to the subject invention.

As used herein, the term “administering” illustratively includes delivery of a molecule such as a protein, polypeptide, or oligonucleotide to a subject by a route illustratively including intravitreal injection, subconjuctival injection, sub-tenon injection, retrobulbar injection, suprachoroidal injection, surgical implantation, topical administration, iontophoretic delivery, oral, rectal, parenteral, intravenous, intramuscular, subcutaneous, intracisternal, intravaginal, intraperitoneal, intravesical, intraventricular, intracranial, intratumoral, other local, transdermal, intrabuccal, intranasal, intrathecal, modifications thereof, or combinations thereof.

An inventive process illustratively includes administering factor V protein, or an oligonucleotide encoding factor V to a subject. A factor V protein is illustratively wild-type factor V with a sequence derived from a mammal. Illustratively, a polypeptide is human with the sequence found at NCBI accession number NP_{—}000121, the contents of which are incorporated herein by reference. Optionally, FV is the bovine sequence found at accession number AAA30512, the contents of which are incorporated herein by reference. Factor V protein is illustratively those having the amino acid sequence disclosed in Cripe et al. (Biochemistry 31:3777, 1992), Jenny et al. (PNAS 84: 4846, 1987), Kane et al. (Biochemistry 26: 6508, 1987), and Kane & Davie (PNAS 83: 6800, 1986) (wild-type human Factor V) or those described in U.S. Pat. No. 7,125,846, the contents of each of which are incorporated herein by reference.

As used herein, the term “factor V” is either a factor V protein or nucleic acid with a wild type sequence, or optionally is a variant illustratively including protein with a substitution, deletion, addition, or modification not normally found in a factor V isolated from a wild-type subject, or nucleic acid sequences that encode variants of wild-type factor V. A factor V variant is optionally B-domainless factor V. A factor V variant is optionally 50%, 55%, 60%, 65%, 70%, 75%, 80%, 85%, 90%, 95%, 99% or other number less than 100% identical to a wild-type factor V.

As used herein, “factor V protein” encompasses, without limitation, factor V, as well as factor V-related proteins including variants of factor V or factor Va. The term “factor V” is may encompass, without limitation, proteins having the amino acid sequence as described in Cripe et al., Biochemistry, 1992 31:3777; Jenny et al., PNAS USA, 1987; 84:4846, Kane et al., Biochemistry, 1987; 26:6508; and Kane & Davie, PNAS USA, 1986; 83:6800 (wild-type human factor V), as well as wild-type factor V derived from other species, the contents of each of these publications are incorporated herein by reference. The term factor V further encompasses natural allelic variations of factor V that may exist and occur from one individual to another. Also, degree and location of glycosylation or other post-translational modifications may vary depending on the chosen host cells and the nature of the host cellular environment. The term “factor V” is also intended to encompass factor V proteins in their zymogen form, as well as those that have been processed to yield their respective bioactive forms. It is appreciated that wild-type factor V protein optionally is the sequence from organisms other than a human illustratively including, but not limited to murine, equine, sheep, goat, guinea pig, pig, or other mammal, or salmon factor V. The specification is directed to factor V from homo sapiens, or variants thereof for illustrative purposes only and is not meant to be a limitation of the factor V sequence operable herein.

Factor V protein or variants thereof are optionally purified from an organism or are recombinant. The term “purified” or “isolated” as used herein, is intended to refer to a composition, isolatable from other components, wherein the protein or peptide is purified to any degree relative to its naturally-obtainable state, i.e., in this case, relative to its purity within plasma or whole blood. An isolated protein or peptide is substantially free from red blood cells, platelets, lymphocytes, or from the full complement of plasma cells and molecules. An isolated protein or peptide, therefore, also refers to a composition, free from the environment in which it may naturally occur.

Generally, “purified” or “isolated” will refer to a protein or peptide composition that has been subjected to fractionation to remove various other components, and which composition substantially retains its expressed biological activity or in the case of factor V, is substantially able to be converted into a protein with at least a portion of the cofactor activity of factor Va. Where the term “substantially” purified is used, this designation refers to a composition in which the protein or peptide forms the major component of the composition, such as constituting about 50% or more of the proteins in the composition.

Various methods for quantifying the degree of purification of the protein or peptide will be known to those of skill in the art in light of the present disclosure as based on knowledge in the art. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide. Illustrative methods for purification of factor V include those described by Katzmann, J. A., et al., Proc. Natl. Acad. Sci. U. S. A., 1981; 78:162-166, and Nesheim, M. E., et al., Methods Enzymol., 1981; 80:249-274, and Gould, W R, et al., J Biol Chem., 2004; 279(4):2383-2393, the contents of each of which are incorporated herein by reference.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater -fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

Factor V protein or variants thereof are optionally isolated from a host cell or host cell medium such as when factor V or variants thereof are recombinantly expressed. Methods of protein isolation illustratively include column chromatography, affinity chromatography, gel electrophoresis, filtration, or other methods known in the art. In some embodiments, factor V or a variant thereof is expressed with a tag operable for affinity purification. A tag is optionally a 6× His tag. A 6× His tagged protein is illustratively purified by Ni-NTA column chromatography or using an anti-6× His tag antibody fused to a solid support. (Geneway Biotech, San Diego, Calif.) Other tags and purification systems are similarly operable.

Factor V or a variant thereof is optionally chemically synthesized. Methods of chemical synthesis have produced proteins greater than 600 amino acids in length with or without the inclusion of modifications such as glycosylation and phosphorylation. Methods of chemical protein and peptide synthesis illustratively include solid phase protein chemical synthesis. Illustrative methods of chemical protein synthesis are reviewed by Miranda, L P, Peptide Science, 2000, 55:217-26 and Kochendoerfer G G, Curr Opin Drug Discov Devel. 2001; 4(2):205-14, the contents of which are incorporated herein by reference.

The inventive factor V protein may also be modified by other techniques, illustratively including denaturation with heat and/or SDS.

The terms “biologically active peptide” and “peptide therapeutic agent,” “peptide,” “polypeptide,” and “protein” are synonymous as used herein and are intended to mean a natural or synthetic compound containing two or more amino acids. Amino acids present in a biologically active peptide include the common amino acids alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine as well as less common naturally occurring amino acids, modified amino acids or synthetic compounds, such as alpha-asparagine, 2-aminobutanoic acid or 2-aminobutyric acid, 4-aminobutyric acid, 2-aminocapric acid (2-aminodecanoic acid), 6-aminocaproic acid, alpha-glutamine, 2-aminoheptanoic acid, 6-aminohexanoic acid, alpha-aminoisobutyric acid (2-aminoalanine), 3-aminoisobutyric acid, beta-alanine, allo-hydroxylysine, allo-isoleucine, 4-amino-7-methylheptanoic acid, 4-amino-5-phenylpentanoic acid, 2-aminopimelic acid, gamma-amino-beta-hydroxybenzenepentanoic acid, 2-aminosuberic acid, 2-carboxyazetidine, beta-alanine, beta-aspartic acid, biphenylalanine, 3,6-diaminohexanoic acid, butanoic acid, cyclobutyl alanine, cyclohexylalanine, cyclohexylglycine, N5-aminocarbonylornithine, cyclopentyl alanine, cyclopropyl alanine, 3-sulfoalanine, 2,4-diaminobutanoic acid, diaminopropionic acid, 2,4-diaminobutyric acid, diphenyl alanine, N,N-dimethylglycine, diaminopimelic acid, 2,3-diaminopropanoic acid, S-ethylthiocysteine, N-ethylasparagine, N-ethylglycine, 4-aza-phenylalanine, 4-fluoro-phenylalanine, gamma-glutamic acid, gamma-carboxyglutamic acid, hydroxyacetic acid, pyroglutamic acid, homoarginine, homocysteic acid, homocysteine, homohistidine, 2-hydroxyisovaleric acid, homophenylalanine, homoleucine, homoproline, homoserine, homoserine, 2-hydroxypentanoic acid, 5-hydroxylysine, 4-hydroxyproline, 2-carboxyoctahydroindole, 3-carboxyisoquinoline, isovaline, 2-hydroxypropanoic acid (lactic acid), mercaptoacetic acid, mercaptobutanoic acid, sarcosine, 4-methyl-3-hydroxyproline, mercaptopropanoic acid, norleucine, nipecotic acid, nortyrosine, norvaline, omega-amino acid, ornithine, penicillamine (3-mercaptovaline), 2-phenylglycine, 2-carboxypiperidine, sarcosine (N-methylglycine), 2-amino-3-(4-sulfophenyl)propionic acid, 1-amino-1-carboxycyclopentane, 3-thienylalanine, epsilon-N-trimethyllysine, 3-thiazolylalanine, thiazolidine 4-carboxylic acid, alpha-amino-2,4-dioxopyrimidinepropanoic acid, and 2-naphthylalanine. Accordingly, the term “biologically active peptide ” as used herein includes peptides having between 2 and about 3000 amino acids or having a molecular weight in the range of about 150-350,000 Daltons.

As used herein the term “variant” is defined as a factor V protein or nucleic acid with a non-wild type substitution, shift, deletion, insertion, phosphorylation, glycosylation, or other modification. A variant is also defined as a fragment of factor V. A variant of factor V is optionally a factor V protein that has or is cleavable to produce a factor V protein with biological activity. Illustratively, a fragment is a truncated version of factor V. Illustratively, a truncated version of factor V is missing one or more N- or C-terminal amino acids or a 5′ or 3′ nucleotide. Optionally, a variant is a factor V with an internal sequence deletion illustratively, a B-domain deleted or fraction of B-domain missing factor V molecule. A variant is optionally resistant to cleavage or other degradation by a protease, other molecule, chemical, or environmental condition such as heat, cold, pH, solvent type, salt concentration, and the like.

As used herein, factor V variants include, without limitation, polypeptides that are or are not precursors for proteins that exhibit substantially the same or improved biological activity relative to wild-type human factor Va, as well as polypeptides, in which the factor V biological activity has been substantially modified or reduced relative to the activity of wild-type human factor Va. These polypeptides include, without limitation, factor V that has been chemically modified and factor V variants into which specific amino acid sequence alterations have been introduced that modify or disrupt the bioactivity of the polypeptide, as well as polypeptides with a slightly modified amino acid sequence, for instance, polypeptides having a modified N-terminal end including N-terminal amino acid deletions or additions, and/or polypeptides that have been chemically modified relative to human factor V.

Factor V-related polypeptides, including variants, encompass those that exhibit at least about 10%, at least about 20%, at least about 30%, at least about 40%, at least about 50%, at least about 60%, at least about 70%, at least about 80%, at least about 90%, at least about 100%, at least about 110%, at least about 120%, and at least about 130%, of the specific activity of wild-type factor V that has been produced in the same cell type, when tested in the factor V activity assay as described in the present specification or otherwise known in the art. Factor V biological activity is illustratively quantified by measuring the ability of a factor V preparation to clot plasma, as described, e.g., in Thorelli et al., Thromb Haemost, 1998; 80:92 et seq., the contents of which are incorporated herein by reference. In this assay, biological activity is expressed as the reduction in clotting time relative to a control sample.

Examples of Factor V equivalents include plasma-derived human Factor V as described, e.g., in Dahlback, J. Clin. Invest., 1980; 66:583-591; Kane & Majerus, J. Biol. Chem., 1981; 256:1002-1007; or Katzmann et al., P.N.A.S., USA, 1981; 78:162-166; recombinant human Factor V as described, e.g., in Kane & Davie, P.N.A.S., USA, 1986, 83:6800-6804; or Jenny et al., P.N.A.S., USA, 1987; 84:4846-4850; platelet-derived Factor V as described, e.g., in Tracy et al., Blood, 1982; 60:59-63, or in Tracy et al., J. Biol. Chem., 1985; 260:2119-2124, bovine Factor V as described, e.g. in Nesheim et al., J. Biol. Chem., 1979; 254:508-517 or Esmon, J. Biol. Chem., 1979; 254:964-973, the contents of each of which are incorporated herein by reference.

Optionally, the ratio of activity of factor V to a factor V equivalent or variant is between 0.1 and 4.0. Optionally, the ratio is at least 1.25, at least 2.0, or at least 4.0.

Factor V-related polypeptides also include fragments of factor V or factor V-related polypeptides retaining their characteristic hemostasis-related activity. The hemostasis-related activity of a factor V polypeptide may, for example, be measured using a factor V-activity assay.

As used herein the term “active” when referring to factor V is intended to refer to a protein that possesses, or has the ability to be converted to a protein with, some level of cofactor activity when in combination with factor Xa or other components of the prothrombinase complex. As used with respect to factor Va, the term active is intended to refer to cofactor activity of the protein. As used herein, the term “factor V activity” is intended to encompass cofactor activity of a factor Va molecule, or action of factor V as a substrate for a protease, or target of a transcriptase, polymerase, or other molecule capable of producing, cleaving, phosphorylating, glycosylating, or otherwise modifying factor V or factor Va.

Factor V or variants thereof are optionally inactive. The term “inactive” is defined herein as possessing less than 10% the cofactor activity of thrombin activated wild-type factor Va. Typical preparations of purified factor V possesses in the range of 1% cofactor activity in the absence of any external activation mechanisms such as by thrombin cleavage. Thus, it is envisioned that isolated factor V or variants thereof are optionally administered in an inactive form.

Modifications and changes are optionally made in the structure (primary, secondary, or tertiary) of the factor V protein which are encompassed within the inventive compound that optionally result in a molecule having similar characteristics to the exemplary polypeptides disclosed herein or which result in resistance to cleavage by a protease. It is appreciated that changes in conserved amino acid bases are most likely to impact the activity of the resultant protein or its cleavage by one or more proteases. However, it is further appreciated that changes in amino acids operable for protease interactions, phospholipid interactions, resistance or promotion of protein degradation, intracellular or extracellular trafficking, secretion, protein-protein interaction, post-translational modification such as glycosylation, phosphorylation, sulfation, and the like, optionally result in increased or decreased activity of an inventive protein while retaining some ability to alter or maintain a physiological activity. Certain amino acid substitutions for other amino acids in a sequence are known to occur without appreciable loss of activity.

In making amino acid changes, the hydropathic index of amino acids is optionally considered. According to the present invention, certain amino acids are substituted for other amino acids having a similar hydropathic index. Each amino acid is assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Without intending to be limited to a particular theory, it is believed that the relative hydropathic character of the amino acid determines the secondary structure of the resultant polypeptide, which in turn defines the interaction of the polypeptide with other molecules. It is known in the art that an amino acid may is some embodiments be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides include variants having about 50%, 60%, 70%, 80%, 90%, and 95% sequence identity to the polypeptide of interest.

Factor V protein is optionally administered in a fully or partially active state. Typical activation of factor V occurs by removal of the B-domain by thrombin cleavage at Arg709, Arg1018, and Arg1545. Alternatively, other proteases are able to activate factor V illustratively including Russell's Viper Venom (RVV). Illustratively, Daboia russelli and Daboia lebetina contain a serine protease that specifically activates FV by a single cleavage at Arg1545. Thus, factor V is optionally cleaved at Arg709, Arg1018, and Arg1545 prior to administration.

Alternatively, or in addition, numerous other activated factor V molecules are operable for administration in the inventive processes. Illustrative examples of activated factor V are presented in U.S. Patent Publication No: 2009/0318344, the contents of which are incorporated herein by reference for both the active factor V molecules as well as for methods of their preparation. Optionally, factor V is cleaved at one or more of Arg709, Arg, 1018, or Arg1545 prior to administration. Optionally, factor V is cleaved at just Arg1545 prior to administration. In some embodiments, factor V is cleaved at one or more sites producing an active factor V molecule, subsequently subjected to purification protocols, and then administered to a subject.

A factor V variant is optionally resistant to proteolysis. Resistance to proteolysis is defined as producing a reduced rate of cleavage at one or more locations by a protease, reduction in binding affinity for a protease, or combinations thereof. As an illustrative example, an amino acid substitution is present in factor V that renders the protein resistant to proteolysis by thrombin, factor Xa, plasmin, trypsin, chymotrypsin, activated protein C, RVV or other protease.

Optionally, an amino acid substitution is at or near a cleavage site of a protease. A protease cleavage site is optionally a cleavage site in factor V or factor Va for plasmin, cathepsin G, elastase, a platelet derived protease, or activated protein C. Plasmin cleaves factor V or factor Va C-terminal to amino acid positions 8, 93, 109, 364, 456, 1545, 1656, 1765, and 1827. (See e.g. Zeibdawi, A, and Pryzdial, E., J. Biol. Chem., 2001; 276(23):19929-36, the contents of which are incorporated herein by reference.) As such, substitution, modification, or deletion of amino acids at or near positions 8, 93, 109, 364, 456, 1545, 1656, 1765, and 1827 render factor V or factor Va resistant to cleavage by plasmin. Activated protein C (APC) cleaves factor Va C-terminal to positions 306, 506, and 679. As such, substitution, modification, or deletion of Arg306, Arg506, Arg679, or nearby residues renders factor V or factor Va resistant to cleavage by APC. A platelet derived protease is illustratively described by Kane, W H, et al., J. Clin. Invest., 1982; 70:1092-1100, and fragments of factor V described by Monkovic, D, and Tracy, P B, J Biol Chem, 1990; 265:17132-17140, the contents of each of which are incorporated herein by reference. Amino acid substitution, modification, deletion, or other at any one of these or other sites will illustratively render the resulting factor V variant resistant to proteolysis. Illustrative examples of amino acid substitutions are described herein or are otherwise known in the art. Illustratively, substituting an Arg with Gly, Ala, Lys or other amino acid will render factor V resistant to protease cleavage at the site of the substitution or related to the site of mutation. It is appreciated the determining whether any particular mutation renders factor V or factor Va resistant to proteolysis is routine in the art. Illustratively factor V is exposed to a protease for 1 min to 24 hours, the reaction is quenched, and the resulting solution is analyzed for cleavage by western blotting methods.

It is appreciated that variants of factor V that are not wild-type at sites other than protease cleavage sites may similarly render the molecule resistant to proteolysis. Illustratively, varying factor V at a site responsible for interactions with a protease will affect binding interactions with the protease decreasing the ability of the protease to cleave factor V. Optionally, a variant position is responsible for promoting tertiary structure of factor V whereby its variation alters the structure reducing the ability of the protease to bind or cleave factor V or alter the association of a cofactor such as heparin or protein S with a protease. As such, a factor V variant is any variant illustratively including one or more amino acid substitutions, modifications, deletions, or additions at a cleavage site or remote from a cleavage site are envisioned as producing a factor V variant that is resistant to proteolysis. Illustrative examples of factor V molecules resistant to APC cleavage by mutation at regions remote from the APC cleavage sites are described by Segers, K., et al., J Biol Chem., 2008; 283(33):22573-81, the contents of which are incorporated herein by reference.

An inventive process includes administering a factor V protein or a variant thereof, or an oligonucleotide encoding factor V or a variant thereof to a subject. A therapeutically effective amount of factor V is administered. A therapeutically effective amount is defined as an amount of an inventive compound that when administered to a subject, ameliorates a condition or symptom of TIC, alters a coagulation response in a subject or in a biological sample obtained from a subject, or increases circulating factor V levels in the whole blood or plasma of a subject. Illustratively, a symptom is bleeding from the skin, mucosa, joints, muscle, genitourinary tract, gastrointestinal tract, or ocular environment; or bleeding at any other internal location or plurality of locations; elevated clot time relative to normal; reduced thrombin potential relative to normal; reduced clot strength; or prolonged clot dissolution time. An effective amount is optionally an amount to increase the level of factor V in the plasma of the subject following administration. Illustratively, the level of factor V is raised to above 1%, 5%, 10%, or 30%, of normal or more. Illustratively, the level of factor V is raised in the plasma to such a level that the subject is free from life-threatening bleeding events.

A therapeutically effective amount of factor V, or a variant thereof is illustratively sufficient to alter a subject's clot time, increase clot strength, prolong clot dissolution time, increase the rate of thrombin generation, increase the amount of thrombin produced, or combinations thereof relative to the absence of such administration. One of ordinary skill in the art recognizes how to measure such parameters. Illustratively, clot time is measured using plasma obtained from a subject following venipuncture and centrifugation by prothrombin time (PT), thrombin time, reptilase time, or activated partial thromboplastin time (aPTT), among others. Methods for performing such assays are known to those of skill in the art. aPTT is illustratively the activated partial thromboplastin time as described by, e.g., Proctor R R, Rapaport S I, Am J Clin Pathol, 1961; 36:212, the contents of which are incorporated herein by reference. Reagents for performing such assays are illustratively obtained from Sigma-Aldrich, St. Louis, Mo. Measurements of clot strength are illustratively performed using a thromboelastograph essentially as described by Glidden, P, et al., Clin Appl Thromb Hemost., 2000; 6:226-233, incorporated herein by reference, or by other methods illustratively those described in U.S. Pat. No. 3,854,324, the contents of which are incorporated herein by reference. Measurements of clot strength are also measurable by determining clot elastic modulus or clot retraction force such as by methods described in U.S. Pat. No. 5,293,772, the contents of which are incorporated herein by reference. U.S. Pat. No. 5,293,772 also describes illustrative methods for measuring clot dissolution time and is similarly incorporated herein by reference for this teaching. Clot lysis is also measurable in vivo illustratively by methods described in U.S. Pat. Publication No. 2010/0063414, the contents of which are incorporated herein by reference. Other methods illustratively include those described by Ghazali, M, and Hayward, G., Anal Bioanal Chem, 2008; 392:897-902, and U.S. Pat. No. 4,276,383 the contents of which are incorporated herein by reference.

Measurements of thrombin generation are illustratively achieved by identification of thrombin-antithrombin complexes essentially as described by Brummel-Ziedins K E, et al, J Thromb Haemost., 2004; 2(2):281-8, the contents of which are incorporated herein by reference. Optionally, measurements of thrombin activity are used to determine the rate and extent of thrombin generation in a sample essentially as described by Gould, W R, et al., Biochemistry, 2005; 44: 9280-9; Gould W R, et al., J Thromb Haemost., 2006, 4: 834-841, or Hemker H C, Beguin S., Thromb Haemost, 2000; 84: 747-51, the contents of each of which are incorporated herein by reference.

A therapeutically effective amount is illustratively dosages of between 10-1000 μg/kg. Optionally, a dose is between about 10-350 μg/kg. In some embodiments, a dose is between 10 and 75 μg/kg. Optionally, a 40 μg/kg dose of the variant factor V polypeptide is used. Patients are illustratively treated immediately upon presentation at the clinic with a bleed. Alternatively, patients may receive a bolus infusion every one to three hours, or if sufficient improvement is observed, a once daily infusion of factor V or variant thereof.

Factor V or variant thereof is optionally administered to a patient via infusion such as by intravenous injection, in a biologically compatible carrier. The factor V or variant thereof is optionally be encapsulated into liposomes or mixed with other phospholipids or micelles to increase stability of the molecule. The factor V or variant thereof is optionally administered alone or in combination with one or more other agents known to modulate hemostasis (e.g., Factor VIIa, FIX, FVIII or FX/Xa and derivatives thereof). In some embodiments, factor V or a variant thereof is the only modulator of hemostasis administered. In some embodiments, factor V or a variant thereof is coadministered with either FIIa, FVIII, FX, or FXa. An appropriate composition in which to deliver factor V or variant thereof may be determined by a medical practitioner upon consideration of a variety of physiological variables, including, but not limited to, the patient's condition and hemodynamic state. A variety of compositions well suited for different applications and routes of administration are well known in the art.

Factor V or variant thereof optionally contains a physiologically acceptable matrix and is formulated as a pharmaceutical preparation. The preparation is optionally formulated using substantially known prior art methods, it can be mixed with a buffer containing salts, such as NaCl, CaCl₂, and amino acids, such as glycine and/or lysine, and in a pH range from 6 to 8. Until needed, the factor V or variant thereof can be stored in the form of a finished solution or in lyophilized or deep-frozen form. Alternatively, the preparation according to the present invention can also be made available as a liquid preparation or as a liquid that is deep-frozen. The preparation according to the present invention is especially stable, i.e., it can be allowed to stand in dissolved form for a prolonged time prior to application.

Also provided is an oligonucleotide encoding factor V or a variant thereof. Illustrative examples of an oligonucleotide or variant thereof are found at accession no: NM_{—}000130, NM_{—}007976, or those encoding the polypeptides described in U.S. Patent Publication No: 2009/0318344, the contents of each of which are incorporated herein by reference.

The term “nucleotide” is intended to mean a base-sugar-phosphate combination either natural or synthetic, linear, circular and sequential arrays of nucleotides and nucleosides, e.g. cDNA, genomic DNA, mRNA, and RNA, oligonucleotides, oligonucleosides, and derivatives thereof. Included in this definition are modified nucleotides which include additions to the sugar-phosphate groups as well as to the bases.

The terms “oligonucleotide” or “nucleic acid” refer to multiple nucleotides attached in the form of a single or double stranded polynucleotide that can be natural, or derived synthetically, enzymatically, and by cloning methods. The terms “nucleic acid” and “oligonucleotide” may be used interchangeably in this application.

An oligonucleotide encodes either wild-type factor V from humans or primates, bovine, murine, equine, pig, or other organism. An oligonucleotide optionally encodes a fragment of factor V. A fragment of factor V is illustratively B-domainless factor V or other protein fragment described herein. An oligonucleotide optionally encodes a variant of factor V. Any protein variant described herein or envisioned by one of skill in the art is encodable by an oligonucleotide operable herein. Illustratively an oligonucleotide encodes a factor V that is resistant to proteolysis. Illustratively, an oligonucleotide encodes a factor V that is resistant to proteolysis by plasmin, cathepsin G, elastase, a platelet derived protease, APC, or combinations thereof. Factor V variants may be produced by means of site-directed mutagenesis or alanine-scanning mutagenesis (see, e.g., Cunningham and Wells, Science, 1989; 244:1081-1085, the contents of which are incorporated herein by reference).

An oligonucleotide is optionally isolated from the cellular materials with which it is naturally associated. Numerous methods are known in the art for the synthesis and production of nucleic acid sequences illustratively including cloning and expression in cells such as E. coli, insect cells such as Sf9 cells, yeast, and mammalian cell types such as Hela cells, Chinese hamster ovary cells, or other cells systems known in the art as amendable to transfection and nucleic acid and/or protein expression. Methods of nucleic acid isolation are similarly recognized in the art. Illustratively, plasmid DNA amplified in E. coli is cleaved by suitable restriction enzymes such as NdeI and XhoI to linearize factor V encoding DNA. The factor V encoding DNA is subsequently isolated following gel electrophoresis using a S.N.A.P.™ UV-Free Gel Purification Kit (Invitrogen, Carlsbad, Calif.) as per the manufacturer's instructions.

Numerous agents are amenable to facilitate cell transfection illustratively including synthetic or natural transfection agents such as LIPOFECTIN, baculovirus, naked plasmid or other DNA, or other systems known in the art.

The nucleic acid sequences of the invention may be isolated by conventional uses of polymerase chain reaction or cloning techniques such as those described in conventional texts. For example, the nucleic acid sequences of this invention may be prepared or isolated from DNA using DNA primers and probes and PCR techniques. Alternatively, the inventive factor V nucleic acid sequences may be obtained from gene banks derived from mus musculus whole genomic DNA or from libraries of human DNA. These sequences, fragments thereof, modifications thereto and the full-length sequences may be constructed recombinantly using conventional genetic engineering or chemical synthesis techniques or PCR, and the like.

It may be more convenient to employ as the recombinant oligonucleotide a cDNA version of the oligonucleotide. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the inventor does not exclude the possibility of employing a genomic version of a particular gene where desired.

As used herein, the terms “engineered” and “recombinant” cells are synonymous with “host” cells and are intended to refer to a cell into which an exogenous DNA segment or gene, such as a cDNA or gene has been introduced. Therefore, engineered cells are distinguishable from naturally occurring cells which do not contain a recombinantly introduced exogenous DNA segment or gene. A host cell is optionally a naturally occurring cell that is transformed with an exogenous DNA segment or gene or a cell that is not modified. A host cell preferably does not possess a naturally occurring gene encoding factor V. Engineered cells are thus cells having a gene or genes introduced through the hand of man. Recombinant cells include those having an introduced cDNA or genomic DNA, and also include genes positioned adjacent to a promoter not naturally associated with the particular introduced gene.

To express a recombinant encoded polypeptide in accordance with the present invention one would prepare an expression vector that comprises a polynucleotide under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli .chi. 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda may be utilized in making a recombinant phage vector that can be used to transform host cells, such as E. coli LE392.

Further useful vectors include pIN vectors and pGEX vectors, for use in generating glutathione S-transferase (GST) soluble fusion proteins for later purification and separation or cleavage. Other suitable fusion proteins are those with β-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to microorganisms, cultures of cells derived from multicellular organisms may also be used as hosts. In principle, any such cell culture is operable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autographica californica nuclear polyhedrosis virus (AcNPV) is used as a vector to express foreign genes. The virus grows in Spodoptera frugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 by sequence extending from the HindIII site toward the Bgll site located in the viral origin of replication.

Viral vectors which are illustratively used in the present invention include, but are not limited to, adenoviral vectors (with or without tissue specific promoters/enhancers), adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors. For a more detailed discussion of the use of adenovirus vectors utilized for gene therapy, see Berkner, 1988, Biotechniques 6:616-629 and Trapnell, 1993, Advanced Drug Delivery Reviews 12:185-199, the contents of which are incorporated herein by reference.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

A vector that can deliver multiple copies of a factor V or variant thereof gene and hence greater amounts of the product of that gene is desirable. Improved adenoviral vectors and methods for producing these vectors have been described in detail in a number of references, patents, and patent applications, including: Mitani and Kubo, Curr Gene Ther. 2002; 2(2):135-44); Olmsted-Davis et al., Hum Gene Ther., 2002; 13(11):1337-47); Reynolds et al., Nat. Biotechnol., 2001; 19(9):838-42); U.S. Pat. No. 5,998,205 (wherein tumor-specific replicating vectors comprising multiple DNA copies are provided); U.S. Pat. No. 6,228,646 (wherein helper-free, totally defective adenovirus vectors are described); 6,093,699 (wherein vectors and methods for gene therapy are provided); U.S. Pat. No. 6,100,242 (wherein a transgene-inserted replication defective adenovirus vector was used effectively in in vivo gene therapy of peripheral vascular disease and heart disease); and International Patent Application Nos. WO 94/17810 and WO 94/23744. Vectors and methods particularly suitable for use with the subject invention are described in U.S. Pat. Application No: 2009/0318344. The contents of each of these publications are incorporated herein by reference for this and the entirety of their teaching.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, anti-metabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin. It is appreciated that numerous other selection systems are known in the art that are similarly operable in the present invention.

It is contemplated that the isolated nucleic acids of the disclosure may be “overexpressed”, i.e., expressed in increased levels relative to its natural expression in cells of its indigenous organism, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or immunoblotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

The proteins and nucleic acid sequences or anti-sense sequences of the invention, alone or in combination with other antigens, antibodies, nucleic acid sequences or anti-sense sequences may further be used in therapeutic compositions and in methods for treating humans and/or animals with TIC, particularly factor V consumptive TIC. For example, one such therapeutic composition may be formulated to contain a carrier or diluent and one or more factor V proteins, protein fragments, or variants of the invention. Suitable pharmaceutically acceptable carriers facilitate administration of the proteins or nucleic acids but are physiologically inert and/or nonharmful.

Formulations optionally include one or more diluents, emulsifiers, preservatives, buffers, excipients, etc. and may be provided in such forms as liquids, powders, emulsions, controlled release, etc. One skilled in the art may formulate the compositions of the invention an appropriate manner, and in accordance with accepted practices, such as those disclosed in Remington's Pharmaceutical Sciences, Gennaro, ed., Mack Publishing Co., Easton, Pa., 1990, the contents of which are incorporated herein by reference. Thus, a typical pharmaceutical composition for intravenous infusion could be made up to contain 250 ml of sterile Ringer's solution and 10 mg of the preparation.

Carriers may be selected by one of skill in the art. Exemplary carriers include sterile water or saline, lactose, sucrose, calcium phosphate, gelatin, dextran, agar, pectin, peanut oil, olive oil, sesame oil, and water. Additionally, the carrier or diluent may include a time delay material, such as glycerol monostearate or glycerol distearate alone or with a wax. In addition, slow release polymer formulations can be used.

Optionally, the inventive composition may also contain conventional pharmaceutical ingredients, such as preservatives, or chemical stabilizers. Suitable ingredients operable herein include, for example, casamino acids, sucrose, gelatin, phenol red, N-Z amine, monopotassium diphosphate, lactose, lactalbumin hydrolysate, and dried milk.

An inventive process optionally includes obtaining a biological sample from a subject. A biological sample is optionally obtained from a subject following trauma, prior to trauma, during trauma, or combinations thereof. As used herein, the term “sample” is defined as a sample obtained from a biological organism, a tissue, cell, cell culture medium, or any medium suitable for mimicking biological conditions. Non-limiting examples of a sample include whole blood, plasma, vascular tissue, liver tissue, neuronal tissue, or combinations thereof.

A biological sample is optionally processed. Processing optionally includes centrifugation and sequestering plasma from whole blood. Methods, instrumentation, and reagents used for obtaining plasma from whole blood are known to those of skill in the art. Optionally, processing includes combining a biological sample with an anticoagulant, illustratively sodium citrate, EDTA, Acid Citrate Dextrose, Heparin, other anticoagulants known in the art, or combinations thereof.

In some embodiments, an inventive process optionally includes obtaining a biological sample from a subject following trauma. The biological sample is optionally analyzed for the presence or absence of factor V protein, or factor V protein fragments in the biological sample. Optionally, whole blood is obtained following trauma. Plasma is prepared by centrifugation, and separated from the red blood cell component.

Immunotechniques such as ELISA or western blot are illustratively used to quantify factor V present in the subject's plasma. Antibodies useful for ELISA or western blot detection are illustratively available from Haematologic Technologies, Essex Junction, VT. ELISA kits for detecting and quantifying the level of factor V are illustratively available from Aniara, Mason, Ohio. Western blotting methods for the detection of fragments of factor V are illustratively described by Camire, R M, et al., Biochemistry, 1998; 37(34):11896-906, the contents of which are incorporated herein by reference. Alternatively, or in addition intact factor V levels are quantified by determining the factor V specific activity in a biological sample. One such method is that described by Lee, C D, and Mann, K G, Blood, 1989; 73:185-190; and Nesheim, M E, et al., Meth. Enzymol., 1981; 80:249 et seq., the contents of each of which are incorporated herein by reference.

Intact factor V in a biological sample obtained following trauma (post trauma factor V value) is illustratively compared to a level of factor V in a similar biological sample from the subject obtained prior to trauma (baseline factor V value). Optionally, the level of intact factor V in a biological sample obtained following trauma is compared to a standard level of factor V (standard factor V value), optionally, in the plasma. Standard factor V values are 7.0 μg/ml with a healthy normal range from about 4 μg/ml to 14 μg/ml. Tracy, P B et al., Blood, 1982; 60: 59-63, the contents of which are incorporated herein by reference this teaching as well as for methods of quantifying factor V in plasma and platelets. Typically, levels of factor V less than 10 percent of normal will have bleeding complications. In some instances levels less than about 30% will lead to bleeding complications. Subjects presenting with TIC typically have less than 10%, more typically less than 1% intact factor V antigen in the plasma.

Quantifying factor V levels in the plasma is an exemplary method of diagnosing factor V consumptive TIC when other factors are present illustratively, the presence of a recent or prior trauma, DIC, hemorrhage, or other symptom of TIC. Factor V consumptive TIC is diagnosed by the presence of one or more symptoms of TIC as well as a level of plasma-derived factor V of less than 30% that of normal or relative to the same patient prior to trauma. One illustrative method of diagnosing trauma induced factor V consumptive coagulopathy is by comparing the level of factor V in a sample from a subject during or following trauma with a level of factor V in an analogous sample from the same subject prior to trauma (baseline factor V value) or to a standard factor V value. Comparing is optionally performed by dividing the post-trauma factor V value by either a baseline factor V value or a standard factor V value to produce a trauma induced factor V ratio. A post trauma factor V level of 30% of normal equates to a post trauma factor V ratio of 0.3. As such, trauma induced factor V consumptive coagulopathy is optionally diagnosed by a post trauma factor V ratio of 0.3 or less, optionally, 0.2, 0.1, 0.01, or less. With respect to standard factor V values, the levels of factor V antigen are illustratively described by Camire, R. M., et al., Blood, 1998; 92: 3035-3041 and Kamphuisen, P. W., et al., Arteriosclerosis, Thrombosis, and Vascular Biology. 2000; 20:1382, the contents of each of which are incorporated herein by reference.

As such, an inventive method illustratively includes obtaining a pre-trauma biological sample from a subject, illustratively plasma. The level of intact factor V in the plasma is quantified to establish a baseline level for the subject. A second post-trauma biological sample is obtained from the subject during or subsequent to trauma wherein the level of intact post-trauma factor V is quantified. The levels of pre-trauma factor V and post-trauma factor V are compared and the presence of factor V consumptive TIC is diagnosed from the comparing.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to humans or mice, specifically, a person having ordinary skill in the art recognizes that similar techniques and other techniques known in the art readily translate the examples to other organisms. Reagents illustrated herein are commonly cross reactive between mammalian species or alternative reagents with similar properties are commercially available, and a person of ordinary skill in the art readily understands where such reagents may be obtained.

### EXAMPLE 1

Five subjects presenting with heat stroke or severe head injury, and severe bleeding are evaluated for the presence of trauma induced factor V consumptive coagulopathy. The patients presenting with head injury also presented with disseminated intravascular coagulation.

Whole blood (0.9 vol) is collected essentially as described by van der Meer F J, et al., Thromb Haemost, 1997; 78(1):631-635, the contents of which are incorporated herein by reference, from the antecubital vein into Sarstedt Monovette tubes (Nümbrecht, Germany) containing 0.106 M of trisodium citrate (0.1 vol). Plasma is prepared by centrifugation for 10 minutes at 2000×g at room temperature.

Intact Factor V antigen levels are measured by a sandwich-type enzyme linked immunosorbent assay (ELISA) with monoclonal antibodies V-6 and V-9, both with a high affinity for the light chain of activated factor V as described by Kamphuisen P W, et al., Arterioscler Thromb Vasc Biol, 2000; 20(5):1382-6, the contents of which are incorporated herein by reference. Briefly, 96-well plates are coated with 3 mg/mL monoclonal antibody V-6 in 0.1 M NaHCO₃ and 0.5 M NaCl, pH 9.0 and stored overnight at 4° C. Plasma samples are diluted 1/100 to 1/400 in 0.05 M triethanolamine, 0.1 M NaCl, 0.01 M EDTA, and 0.1% Tween-20, pH 7.5, and 100 μL per sample is incubated for 3 hours in the coated wells. Monoclonal antibody V-9, labeled with horseradish peroxidase (Invitrogen, Corp., Carlsbad, Calif.) and diluted to 2 mg/mL in 0.05 M triethanolamine, 0.1 M NaCl, 0.01 M EDTA, and 0.1% Tween-20, pH 7.5, is used for the detection of immobilized factor V (2-hour incubation). Finally, 0.42 mM 3,3′,5,5′-tetramethylbenzidine, 0.1 M sodium acetate, and 1.1 M H₂O₂, pH 5.5, are added, and the reaction was stopped after 20 minutes with 100 μL H₂SO₄. After every step, plates are washed 4 times with 0.05 M triethanolamine, 0.1 M NaCl, and 0.1% Tween-20, pH 7.5. Pooled normal plasma (PNP), prepared from the plasma of 60 healthy volunteers (mean age 40 years) and diluted 1/50 to 1/3200, is used as a reference. Each of the five patients demonstrate less than 5% factor V levels in the plasma.

Plasma samples are also tested for clot time by analyses of aPTT values as compared to normals. Aliquots (55 μl) of factor V (30 nM) in 50 mM Pipes, 100 mM NaCl, 2 mM EDTA, 1% BSA, pH 7.2, are incubated for 5 min with an 55 μl aliquot containing 100 μM PC/PS vesicles and 50 mM CaCl₂ in the same buffer. A 55 μl aliquot of either normal human plasma (NHP) or factor V-deficient plasma is then added and clotting followed for 400 seconds in an ACL clotting machine using the standard APTT program. Each bleeding patient demonstrates a significant prolongation of aPTT.

Additional assays of PT, thrombin time, and thrombin generation are performed. Plasma factor V levels are calculated based on relative clotting time prior to and following administration of aPTT or PT reagent. Clot times indicative of the level of factor V are rapidly achieved and represent a sufficient estimate of plasma factor V levels.

The five patients are each administered a different preparation of factor V protein as described in Table 1:

Factor V preparation

| | |
|---|---|
| Patient 1 | Full length wild-type human factor V |
| Patient 2 | B-domain deleted human factor V |
| Patient 3 | Factor V variant R306A: K456A |
| Patient 4 | Factor V variant R306A: K456A |
| Patient 5 | Factor V variant R506Q |

Each patient is administered the respective factor V preparation with a bolus intravenous dose of 4 mg followed by an intravenous infusion dose of 250 μg/kg/day by intravenous infusion. These numbers are determined to achieve a desired 40% factor V plasma concentration in the patient using the known half life of factor V or 12-36 hours. The frequency of dosing should be adjusted to empirically to achieve proper hemostasis.

Administration of each factor V preparation produces an improvement in clot times. aPTT, PT, and thrombin times are reduced. Similarly, thrombin generation rates are increased. Each patient presents with fewer and less severe bleeding complications.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified. Methods of nucleotide amplification, cell transfection, and protein expression and purification are similarly within the level of skill in the art.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1) A process of treating trauma induced factor V consumptive coagulopathy in a subject comprising:
administering an effective amount of an isolated factor V protein to a subject diagnosed with trauma induced factor V consumptive coagulopathy, whereby said administering alters a symptom of said trauma induced factor V consumptive coagulopathy.

2) The process of claim 1 wherein said administering alters the subject's clot time, increases the magnitude of thrombin generation, increases the rate of thrombin generation, increases clot strength, or prolongs clot dissolution time in said subject's plasma or whole blood.

3) The process of claim 1 wherein said factor V is inactive prior to said administering.

4) The process of claim 1 wherein said factor V is a factor V variant with a non-wild-type amino acid at a protease cleavage site.

5) The process of claim 1 wherein said factor V is a factor V variant with a non-wild-type amino acid substitution, deletion, or addition that renders factor V or factor Va resistant to cleavage by a protease.

6) The process of claim 5 wherein said amino acid substitution, deletion, or addition alters the cleavage rate of factor V or factor Va by plasmin, cathepsin G, elastase, a platelet derived protease, activated protein C, or combinations thereof.

7) The process of claim 5 wherein said amino acid substitution, deletion, or addition is at a cleavage site in factor V or factor Va for plasmin, cathepsin G, elastase, a platelet derived protease, activated protein C, or combinations thereof.

8) The process of claim 1 wherein said factor V is cleaved at Arg 709, Arg 1018, Arg 1545, or combinations thereof, prior to said administration.

9) The process of claim 1 wherein said factor V is recombinantly expressed.

10) The process of claim 1 further comprising:
obtaining a biological sample from said subject following trauma; and
quantifying post-trauma factor V antigen in said biological sample to produce a post-trauma factor V value.

11) The process of claim 10 further comprising:
dividing said post-trauma factor V value by a standard factor V value to obtain a trauma induced factor V ratio; and
diagnosing trauma induced factor V consumptive coagulopathy in said subject from said based on the value of said trauma induced factor V ratio.

12) The process of claim 11 further comprising:
obtaining a first biological sample from said subject prior to trauma;
quantifying pre-trauma factor V antigen in said first biological sample to produce a baseline factor V value;
dividing a post-trauma factor V value by said baseline factor V value to produce a trauma induced factor V ratio; and
diagnosing trauma induced factor V consumptive coagulopathy in said subject from said based on the value of said trauma induced factor V ratio.

13) The process of claim 11 wherein said trauma induced factor V consumptive coagulopathy is diagnosed if said value of said trauma induced factor V ratio is 0.3 or less.

14) The process of claim 12 wherein said trauma induced factor V consumptive coagulopathy is diagnosed if said value of said trauma induced factor V ratio is 0.3 or less.

15) The process of claim 1 wherein said subject has at least one copy of a gene encoding wild-type factor V.

16) A process of treating factor V consumptive coagulopathy in a subject comprising:
administering an effective amount of an isolated factor V variant protein to a subject diagnosed with factor V consumptive coagulopathy, whereby said administering alters a symptom of said trauma induced factor V consumptive coagulopathy.

17) The process of claim 16 wherein said factor V variant is resistant to cleavage by plasmin, cathepsin G, elastase, a platelet derived protease, activated protein C, or combinations thereof.

18) A process of treating factor V consumptive coagulopathy in a subject comprising:
quantifying the level of factor V in blood, or a fraction thereof, from a subject following or during trauma to produce a post-trauma factor V value;
dividing said post-trauma factor V value by a standard factor V value or a baseline factor V value to produce a trauma induced factor V ratio;
diagnosing trauma induced factor V consumptive coagulopathy in said subject based on the value of said trauma induced factor V ratio; and
administering an effective amount of an isolated factor V variant protein to said subject, whereby said administering alters clot time, increases clot strength, or prolongs clot dissolution time.

19) The process of claim 18 wherein said factor V variant protein has a non-wild-type amino acid substitution, deletion, or addition that renders factor V or factor Va resistant to cleavage by a protease.

20) The process of claim 19 wherein said amino acid substitution, deletion, or addition alters the cleavage rate of factor V or factor Va by plasmin, cathepsin G, elastase, a platelet derived protease, activated protein C, or combinations thereof.

21) The process of claim 19 wherein said amino acid substitution, deletion, or addition is at a cleavage site in factor V or factor Va for plasmin, cathepsin G, elastase, a platelet derived protease, activated protein C, or combinations thereof.

22) A process of decreasing clot time in a subject with trauma induced factor V consumptive coagulopathy comprising:
administering an effective amount of an isolated factor V protein to a subject diagnosed with trauma induced factor V consumptive coagulopathy,
whereby said administering decreases clot time of plasma isolated from said subject.

23) The process of claim 22 wherein said factor V has a non-wild-type amino acid substitution, deletion, or addition that renders factor V or factor Va resistant to cleavage by a protease.

24) The process of claim 23 wherein said amino acid substitution, deletion, or addition alters the cleavage rate of factor V or factor Va by plasmin, cathepsin G, elastase, a platelet derived protease, activated protein C, or combinations thereof.
